(19) United States
(12) Patent Application Publication
(10) Pub. No.: US 2010/0227368 A1
(43) Pub. Date: Sep. 9, 2010

(54) RENEWABLE ENERGY SYSTEM

(75) Inventor: Carl A. Steiner, Erie, PA (US)

Correspondence Address: THE WEBB LAW FIRM, P.C., 700 KOPPERS BUILDING, 436 SEVENTH AVENUE, PITTSBURGH, PA, 15219, US

(73) Assignee: TM INDUSTRIAL SUPPLY, INC., Erie, PA (US)

(21) Appl. No.: 12/677,930

(22) PCT Filed: Sep. 19, 2008
(86) PCT No.: PCT/US08/76996

§ 371 Date: Mar. 12, 2010

Related U.S. Application Data
(60) Provisional application No. 60/994,352, filed on Sep. 19, 2007.

(51) Int. Cl. C12P 7/06; C12P 3/00; C12M 1/00

(52) U.S. Cl. 435/161; 435/168; 435/289.1

(57) ABSTRACT

A renewable energy system and related process includes a biomass unit for receiving biomass energy sources, e.g., municipal wastes, used tires and wood waste. The biomass is fed to a power plant which produces electricity and steam which are fed to several units of the system. A bio-diesel unit uses the electricity to convert vegetable oil and animal fat into a bio-diesel fuel. An algae farm uses the electric power and steam in the form of heat to produce algae oil which is fed to the bio-diesel unit and which also is biomass. A corn unit and a corn stover unit produce ethanol. The corn unit also produces corn oil which is fed to the bio-diesel unit, and residue corn becomes dry distilled grains. A fermentation unit discharges carbon dioxide which along with that discharged by the power plant is distributed to the algae farm for growing algae. Also described is a method for converting carbon dioxide to oxygen through the use of algae.

### CROSS REFERENCE TO RELATED APPLICATIONS

The present application claims priority to U.S. Provisional. Application No. 60/994,352 filed on Sep. 19, 2007, which is herein incorporated by reference in its entirety.

### BACKGROUND OF THE INVENTION

1. Field of the Invention

This invention relates to a renewable energy system, and more particularly, to a renewable energy cycle which uses biomass to produce fuels, e.g., ethanol and bio-diesel; chemicals, e.g., glycerin; and dry distilled grains (DDGs) for feeding livestock, e.g., cows. The invention also relates to a process in a renewable energy system.

2. Description of Related Art

Renewable energy processes may involve one or more renewable energy resources, such as solar, wind, water, plants, animals and municipal wastes, since these resources always exist. Renewable energy resources generally offer clean alternatives to fossil fuels, e.g., coal, oil and natural gas, for supplying most energy needs since the renewable energy resources produce very little or no pollution or greenhouse gases.

A prior art renewable energy process uses coal bed methane, coal gasification and land fill gas recovery for biogas production used to produce steam, heat and electricity needed in the cycle. Manure slurries are used to produce dry fertilizer products for fertilizing the corn fields. The corn is used in the production of ethanol and bio-diesel. Such a process is illustrated in the brochure entitled “Where Can Fluid Engineering Help Your Bio-fuel Plant?” provided by Fluid Engineering, Erie, Pa.

Among many things, this illustration shows that carbon dioxide produced within the system can be bubbled into ponds to grow algae that can be converted into bio-diesel fuel, and one source of carbon dioxide is from the fermentation tanks for the corn. Corn is used to produce DDGs for feeding livestock. Corn, corn oil, algae and other products are used to produce bio-diesel which is made through a chemical process called transesterification whereby glycerin is separated from the fat and/or vegetable oils. The transesterification process leaves behind two products—methyl esters which is the chemical name for bio-diesel and glycerin which is used in food and beverages, phalli aceuticals, cosmetics and toiletries, paper and printing, textiles, livestock, and biodegradable packaging.

It can be appreciated that this prior art renewable energy process involves a complicated system and uses coal bed methane, coal gasification, landfill gas recovery and manure slurries as its main input components for producing outputs such as ethanol and bio-diesel.

### SUMMARY OF THE INVENTION

It is therefore an object of the invention to provide a simple compact renewable energy system and related process for producing as outputs ethanol, DDGs and bio-diesel fuel. It is another object of the invention to provide a simple, compact renewable energy system and related process which utilizes biomass as its main input component for generating the required heat, steam and electricity for operating the system and for producing electricity for human consumption. It is still yet another object of the invention to provide a simple, compact renewable energy system and related process which contains several units having one output, which is a final product for human consumption and another output, which is either used to produce the bio-diesel fuel or which is biomass which is fed to the biomass unit. It is therefore a still further object of the invention to provide a simple, compact renewable energy system and related process which is more efficient than prior art processes and systems.

Accordingly, the present invention is directed to an improved renewable energy system and related process for producing outputs such as drain distilled grains (DDGs) for livestock, and bio-diesel fuel, ethanol and electricity for human consumption. The system includes a biomass energy unit, a power plant, a power grid, an algae farm unit, a corn stover unit, a corn unit, a fermentation cycle unit between the corn stover unit and the corn unit and a biomass unit. The system uses biomass as the energy input into the system. Sources for this biomass energy include organic components from municipal wastes, rubber from used tires and hog fuel also known as wood waste which includes agricultural and forestry waste and residue.

This biomass is used directly to produce steam and electricity in a power plant. Most of the electricity is distributed to a power grid for public consumption; however, a small percentage of the electric power along with a great percentage of the steam is distributed to the several units in the system. Specifically, the electric power is distributed to the bio-diesel unit for producing the bio-diesel fuel. The electric power and steam are distributed to the algae farm units to produce oil whereby some of this oil is cycled to the bio-diesel unit and some of the oil is cycled as biomass to the biomass unit. Electric power and steam are distributed to the corn unit and to the corn stover unit to produce ethanol. The fermentation unit produces carbon dioxide which along with the carbon dioxide produced by the power plant is cycled to the algae farm to grow algae. The algae are used to produce an oil that is converted into bio-diesel fuel in the bio-diesel unit as described hereinabove. Some of the by-product from the corn stover unit is used as biomass. Corn oil from the corn unit is fed along with algae oil, animal fat and soybean oil to the bio-diesel unit to produce bio-diesel fuel. Glycerin is extracted from the bio-diesel fuel in the bio-diesel unit and is used in products, such as glycerol soap, or to treat lumber.

The present invention is also directed to a process for a renewable energy system for producing at least bio-diesel fuel and ethanol as end products, the steps including: providing a biomass energy unit for receiving at least one source of biomass energy; converting the biomass energy into electric power and steam in a power plant for receiving the one source of biomass energy; converting vegetable oil and animal fat into bio-diesel fuel in a bio-diesel unit for receiving the electric power from the power plant; producing ethanol and corn oil; in a corn stover unit for receiving the electric power and the steam from the power plant, producing ethanol and biomass in a corn unit for receiving the electric power and the steam from the power plant; distributing the biomass in the corn stover unit to the biomass energy unit; providing a fermentation cycle unit for producing ethanol and corn oil in the corn unit and for producing ethanol and biomass in the corn stover unit; providing an algae farm that receives the electric power and the steam from the power plant for producing algae oil; and distributing the algae oil to the bio-diesel unit for conversion into bio-diesel fuel. The carbon dioxide discharged from the fermentation cycle unit and the power plant is distributed to the algae farm to grow algae, and the corn oil is distributed to the bio-diesel unit where it is converted into bio-diesel fuel.

The present invention is also directed to a method for converting carbon dioxide to oxygen comprising the steps of: a) providing a source of carbon dioxide gas; b) providing algae; c) contacting the carbon dioxide gas with the algae; and d) reacting the algae with the carbon dioxide gas such that the carbon dioxide gas is converted to oxygen and other by-products. In one aspect, the carbon dioxide is produced by a fermentation process, such as the fermentation of corn or other grains. In another aspect, the by-product produced is oil, water or biomass.

The present invention is also directed to an apparatus for producing oxygen comprising: a) a means for providing a source of carbon dioxide gas; b) a means for providing algae; c) a means for contacting the carbon dioxide gas with the algae; and d) a means for reacting the algae with the carbon dioxide gas such that the carbon dioxide gas is converted to oxygen and other by-products. In one embodiment, the means for providing a source of CO₂ gas is a fermentor. In another embodiment, the means for providing algae is an algae farm. In an additional embodiment, the means for contacting the carbon dioxide gas with the algae is an algae pond unit. In another embodiment, the means for reacting the algae with the carbon dioxide gas such that the carbon dioxide gas is converted to oxygen and other by-products is electric power. In still another embodiment, the means for reacting the algae with the carbon dioxide gas such that the carbon dioxide gas is converted to oxygen and other by-products is steam. In one aspect, the fermentor is a grain fermentor, such as a corn fermentor. In another aspect, the by-product produced is oil, water or biomass.

These and other features and characteristics of the present invention, as well as the methods of operation and functions of the related elements of structures and the combination of parts and economies of manufacture, will become more apparent upon consideration of the following description with reference to the accompany drawing, all of which form a part of this specification.

### BRIEF DESCRIPTION OF THE DRAWING

FIG. 1 is a schematic illustrating a renewable energy system of the present invention.

### DESCRIPTION OF THE INVENTION

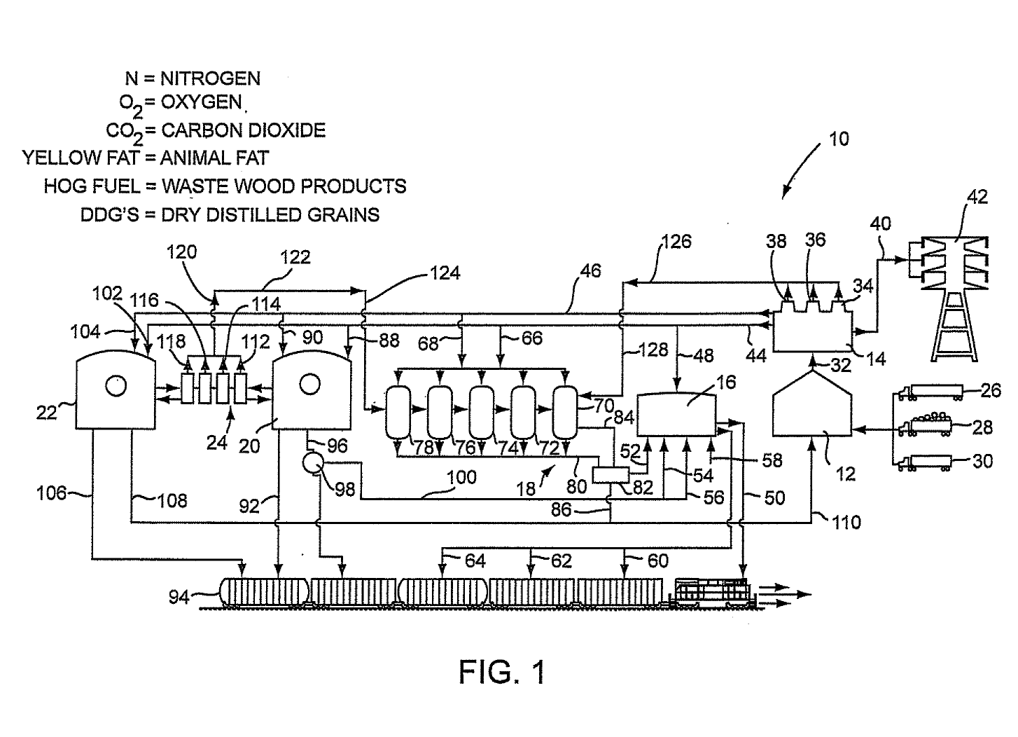

A renewable energy system 10 of FIG. 1 includes a biomass unit 12, a power plant 14, a bio-diesel unit 16, an algae farm 18, a corn unit 20, a corn stover unit 22 and a fermentation cycle unit 24 associated with the corn unit 20 and the corn stover unit 22. The biomass unit 12 is supplied with biomass energy sources, such as organic components from municipal wastes as indicated by reference number 26, rubber from used tires as indicated by reference number 28 and hog fuel also known as wood waste which includes agricultural forestry waste and residue as indicated by reference number 30. Other non-limiting alternative sources of biomass energy may include wood, food crops, e.g., cornhusks, grasses and other plants, organic components from industrial wastes, sewage and methane gas harvested from community landfills. A person of skill in the art would recognize that there are numerous sources of biomass energy. As alluded to hereinabove, biomass is a renewable energy source since trees and crops will continue to grow and waste will continue to exist. This material generally is known to contain residual energy, which can be released by burning it in biomass power plants, such as that indicated by reference number 12 in FIG. 1.

As shown by arrow 32 in FIG. 1, the biomass in biomass unit 12 is fed to the power plant 14 which produces electricity and steam and which discharges exhaust solids and carbon dioxide (CO₂) from stacks 34, 36, and 38. As indicated by an arrow 40 to the right of power plant 14, electricity is provided to a power grid 42 for human use. As indicated by an arrow 44, to the left of power plant 14, electric power is delivered to the several units and as indicated by an arrow 46, to the left of power plant 14, steam is delivered to the several units of system 10 for their operation.

As shown by an arrow 48, electric power is delivered to the bio-diesel unit 16 to produce bio-diesel fuel as indicated by an arrow 50 which can be used directly in any type of diesel engine or can be shipped to domestic and international markets for human use. As shown, the bio-diesel fuel is produced from algae oil as indicated by an arrow 52, animal fat or yellow fat as indicated by an arrow 54, corn oil as indicated by an arrow 56, and soybean oil as indicated by an arrow 58. As discussed hereinabove, bio-diesel is made through a chemical process referred to as transesterification whereby glycerin is separated from the fat and vegetable oils. The glycerin may be used in the manufacture of glycerol soap as shown by an arrow 60, is used to treat lumber as shown by an arrow 62 and is used as bio-diesel as shown by an arrow 64. One of skill in the art would recognize that the glycerin produced by this process may have multiple applications. Bio-diesel fuel has advantages over diesel fuel which is made from fossil fuels, e.g., coal and natural gas, in that it burns cleaner, is renewable and does not cause air pollution. Also, bio-diesel fuel can be made from cooking oil and other types of fresh oils other than those vegetable oils shown in FIG. 1.

To the left of the bio-diesel unit 16 of FIG. 1, and as indicated respectively by arrows 66 and 68, electric power and steam in the form of heat are delivered or supplied from power plant 14 to algae pond units 70, 72, 74, 76 and 78 of algae farm 18. As indicated, algae farm 18 produces more than 30,000 gallons of algae per acre per year. It has recently become appreciated that algae are the ultimate feedstock for producing bio-diesel fuel. As indicated by an arrow 80, these algae are delivered to a separator 82 to produce three outputs, which are oil, water and biomass. Approximately 63% is algae oil, which is fed as indicated by arrow 52 into bio-diesel unit 16, about 10% is water and about 27% is biomass. As indicated by an arrow 84, the water from separator 82 is cycled into the algae pond units, and the biomass as indicated by an arrow 86, along with the biomass from the corn stover unit 22, is fed back to the biomass unit 12, more about which will be discussed hereinbelow.

To the left of algae farm 18 of FIG. 1 and as indicated respectively by arrows 88 and 90, electric power and steam from power plant 14 is delivered to corn unit 20 which are used to produce ethanol and DDGs. This corn unit 20 could produce approximately 50 million gallons of ethanol per year. The amount of ethanol produced is a function of the size of the corn unit 20 and the amount of corn supplied thereto. In one non-limiting embodiment of the invention, as indicated by an arrow 92, the ethanol is delivered to a railroad car 94 which is then shipped for human use. One of skill in the art will recognize that the ethanol can be delivered to and distributed by any acceptable transport system. As indicated by an arrow 96, the DDGs are subjected to an oil extraction device 98 whereby corn oil is extracted from the DDGs. As indicated by arrow 100, and as discussed hereinabove, this corn oil is delivered to the bio-diesel unit 16.

To the left of corn unit 20 and as indicated respectively by arrows 102 and 104, electric power and steam from power plant 14 is delivered to corn stover unit 22 which are used to produce ethanol and biomass as indicated by arrows 106 and 108, respectively. This corn stover unit 22 produces approximately 20 million gallons of ethanol per year. In one non-limiting embodiment, as indicated by an arrow 106, the ethanol is also delivered to railroad car 94 which is then shipped for human use. As is known, corn stover is a by-product of corn and is made from stalks or husks that remain once the corn is harvested. As indicated by an arrow 108, the cornhusks and stalks, now considered as sources of biomass energy, are delivered as indicated by an arrow 110 to the biomass unit 12. The amount of ethanol produced is a function of the size of the corn stover unit 22 and amount of corn stover supplied thereto.

The fermentation cycle unit 24 is located between and is associated with corn unit 20 and the stover unit 22 in FIG. 1. The fermentation unit 24 ferments the corn and the corn stover in the production of ethanol. As indicated by the upward arrows 112, 114, 116 and 118, carbon dioxide is discharged from this fermentation unit 24. As indicated by arrows 120, 122 and 124 leading out of the fermentation unit 24, this carbon dioxide gas is delivered to the algae farm 18 as indicated to the left of algae faun 18. As indicated by arrows 126 and 128 to the right of algae farm 18 and leading out of the exhaust stacks 34, 36 and 38 of power plant 14, the carbon dioxide gas produced from power plant 14 is delivered to the algae farm 18. In a known manner, this carbon dioxide gas is bubbled into the algae ponds 70, 72, 74, 76 and 78 to grow the algae that then are converted into bio-diesel fuel and a discharge or oxygen and oxygen byproducts.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of this description. For example, it is to be understood that the present invention contemplates that, to the extent possible, one or more features of any embodiment can be combined with one or more features of any other embodiment.

The invention claimed is:

1. A renewable energy system for producing at least bio-diesel and ethanol as end products, comprising:
a biomass energy unit for receiving at least one source of biomass energy;
a power plant for receiving said at least one source of biomass energy and for converting this biomass energy into electric power and steam;
a bio-diesel unit for receiving said electric power from said power plant for converting vegetable oil and animal fat into bio-diesel fuel;
acorn unit for receiving said electric power and said steam from said power plant for producing ethanol and corn oil;
a corn stover unit for receiving said electric power and said steam from said power plant for producing ethanol and biomass and which biomass is distributed to said biomass energy unit;
a fermentation cycle unit associated with said corn unit and said corn stover unit for producing said ethanol and said corn oil in said corn unit and for producing said ethanol and said biomass in said corn stover unit; and
an algae farm for receiving said electric power and said steam in the form of heat from said power plant for producing algae oil which is distributed to said bio-diesel unit for conversion into said bio-diesel fuel.

2. The renewable energy system of claim 1, wherein said fermentation cycle unit and said power plant are configured to produce carbon dioxide gas;
wherein said algae farm unit is designed to receive said carbon dioxide gas from said fermentation cycle unit and said power plant and is configured to use said carbon dioxide gas to grow algae; and
wherein said bio-diesel unit is designed to receive said corn oil produced in said corn unit for conversion into said bio-diesel fuel.

3. The renewable energy system of claim 1, wherein said fermentation cycle unit is configured to produce carbon dioxide gas;
wherein said algae farm unit is designed to receive said carbon dioxide gas from said fermentation cycle unit and said power plant is configured to use said carbon dioxide gas to grow algae; and
wherein said bio-diesel unit is designed to receive said corn oil produced in said corn unit for conversion into said bio-diesel fuel.

4. The renewable energy system of claim 1, wherein said power plant is configured to produce carbon dioxide gas;
wherein said algae farm unit is designed to receive said carbon dioxide gas from said fermentation cycle unit and said power plant is configured to use said carbon dioxide gas to grow algae; and
wherein said bio-diesel unit is designed to receive said corn oil produced in said corn unit for conversion into said bio-diesel fuel.

5. A process for a renewable energy system for producing at least bio-diesel fuel and ethanol as end products, the steps comprising:
providing a biomass energy unit for receiving at least one source of biomass energy;
converting said biomass energy into electric power and steam in a power plant for receiving said at least one source of biomass energy;
converting vegetable oil and animal fat into bio-diesel fuel in a bio-diesel unit for receiving said electric power from said power plant;
producing ethanol and corn oil in a corn unit for receiving said electric power and said steam from said power plant;
in a corn stover unit for receiving said electric power and said steam from said power plant, producing ethanol and biomass,
distributing said biomass in said corn stover unit to said biomass energy unit;
providing a fermentation cycle unit for producing said ethanol and said corn oil in said corn unit and for producing said ethanol and said biomass in said corn stover unit;
providing an algae farm for receiving said electric power and said steam in the form of heat from said power plant for producing algae oil; and
distributing said algae oil produced in said algae farm to said bio-diesel unit for conversion into said bio-diesel fuel.

6. The process for a renewable energy system of claim 5, wherein said fermentation cycle unit and said power plant produce carbon dioxide gas, the steps further comprising:
distributing said carbon dioxide gas from said fermentation cycle unit and said power plant to said algae farm unit to grow algae; and
distributing said corn oil produced in said corn unit to said bio-diesel unit for conversion into said bio-diesel fuel.

7. A method for converting carbon dioxide to oxygen comprising the steps of:
a) providing a source of carbon dioxide gas;
b) providing algae;
c) contacting the carbon dioxide gas with the algae; and
d) reacting the algae with the carbon dioxide gas such that the carbon dioxide gas is converted to oxygen and other by-products.

8. The method of claim 7 wherein the carbon dioxide is produced by a fermentation process.

9. The method of claim 8 wherein the fermentation process is the fermentation of grain.

10. The process of claim 9 wherein the fermentation process is the fermentation of corn.

11. The process of claim 7 wherein the by-product produced is oil.

12. The process of claim 7 wherein the by-product produced is water.

13. The process of claim 7 wherein the by-product produced is biomass.

14. An apparatus for producing oxygen comprising:
a) a means for providing a source of carbon dioxide gas;
b) a means for providing algae;
c) a means for contacting the carbon dioxide gas with the algae; and
d) a means for reacting the algae with the carbon dioxide gas such that the carbon dioxide gas is converted to oxygen and other by-products.

15. The apparatus of claim 14 wherein the means for providing a source of CO₂ gas is a fermentor.

16. The apparatus of claim 14 wherein the means for providing algae is an algae farm.

17. The apparatus of claim 14 wherein the means for contacting the carbon dioxide gas with the algae is an algae pond unit.

18. The apparatus of claim 14 wherein the means for reacting the algae with the carbon dioxide gas such that the carbon dioxide gas is converted to oxygen and other by-products is electric power.

19. The apparatus of claim 14 wherein the means for reacting the algae with the carbon dioxide gas such that the carbon dioxide gas is converted to oxygen and other by-products is steam.

20. The apparatus of claim 15 wherein the fermentor is a grain fermenter.

21. The apparatus of claim 20 wherein the grain fermentor is a corn fermentor.

22. The apparatus of claim 14 wherein the by-product produced is oil.

23. The apparatus of claim 14 wherein the by-product produced is water.

24. The apparatus of claim 14 wherein the by-product produced is biomass.
